(19) United States
(12) Patent Application Publication
(10) Pub. No.: US 2009/0105089 A1
(43) Pub. Date: Apr. 23, 2009

(54) METHOD FOR DETECTING INTRACYTOPLASMIC PROTEIN/PROTEIN INTERACTIONS

(75) Inventors: Laurence Renaut, Villefranche De Lauragais (FR); Khalil Bouayadi, Ramonville Saint-Agne (FR)

Correspondence Address: YOUNG & THOMPSON, 209 Madison Street, Suite 500, ALEXANDRIA, VA, 22314, US

(73) Assignee: MILLEGEN, Labege Cedex (FR)

(21) Appl. No.: 12/297,712

(22) PCT Filed: Apr. 18, 2007
(86) PCT No.: PCT/IB2007/001010

§ 371 Date: Oct. 20, 2008

(30) Foreign Application Priority Data
Apr. 19, 2006 (EP) 06290641.7

(51) Int. Cl. C40B 30/06; C12N 15/63; C12N 1/19; C12N 5/00; C12P 21/00; C40B 40/08; C40B 40/02

(52) U.S. Cl. 506/10; 435/320.1; 435/254.2; 435/325; 506/17; 506/14; 435/69.1

(57) ABSTRACT

The present invention provides a versatile and sensitive method for studying interaction of two peptides or polypeptides A and B within the cytoplasm of a host cell. The method is based on the use of two distinct chimeric polypeptides. The first chimeric polypeptide containing an aggregation domain fused to a polypeptide A and the second one containing a phenotype-associated functional domain fused to a second polypeptide B. When these chimeric polypeptides are co-expressed within a host cell allowing aggregation of the aggregation domain, the phenotype of the host cell depends on the entrapment of the phenotype-associated functional domain which only occurs when A and B interact with each other.

### FIELD OF THE INVENTION

This invention relates to kits and methods for detecting and/or selecting a peptide or polypeptide which binds to a bait peptide or polypeptide.

### BACKGROUND OF THE INVENTION

By the end of the human genome sequencing project, several open reading frames coding for new proteins were identified. This finding allowed biological research to focus on the characterisation of these new proteins, involving investigation or identification of interacting protein partners, chemical compounds which disturb protein interaction and specific antibodies for targeting protein.

For this purpose, methods allowing studies of protein/protein interaction have been developed: Different in vitro methods for protein/protein interaction studies such as immunoprecipitation, pull down assays, TAP (Tandem Affinity Purification) and protein array are based on the isolation of interacting proteins. In these methods, proteins of interest, tagged or not, are directly or indirectly immobilised and proteins having interaction properties are retained on their targets.

The major drawback of these methods is that after its isolation, the candidate protein for interaction with the protein of interest must be characterised. For this purpose, long and cost effective methods like 2D SDS-page and mass spectroscopy characterisation have to be used.

In order to facilitate the characterisation of the putative interacting partner, methods allowing the association of candidate protein with their own nucleotide counterparts have been developed.

During the last twenty years, many display methods have been developed and among them, phage display, yeast display, and bacterial display are widely used for screening studies. In these methods, a peptide or a protein is expressed at the surface of the cell or of the virus containing the counterpart genetic information. When interaction occurs, the structure containing the counterpart nucleotide sequence is retained with the target protein and after several cycles of binding, the polypeptide of interest linked with its counterpart nucleotide sequence is enriched and can be characterised. These techniques allow large protein libraries to be screened against a bait protein. However, the bait protein has to be purified. This is a major drawback because purification of proteins can often be very problematic.

Methods allowing the detection of intracellular interaction have been developed. Within these methods, the bait protein is expressed in the cell and purification steps are not required.

The yeast two hybrid method (cf. Fields and Song, 1989 and WO0200729) is based on the transcription of a reporter gene following a protein/protein interaction between two hybrid-proteins. Thus, bait and putative interacting protein partners are fused respectively with the DNA binding domain and the transcription activation domain of a transcription factor. When the interaction occurs, the physical joining of these two domains allows the functional restoration of the transcription factor and the reporter gene expression. However large numbers of controls are required in order to reduce the number of false positive results. Furthermore interactions occur in the nucleus and not all proteins can be studied with this system.

To overcome this drawback, several techniques have been developed in order to highlight protein/protein interaction occurring in the cytoplasm of cells. Among them, the cyto Trap method (cf. Broder et al, 1998 and U.S. Pat. No. 5,776,689) and the protein fragment complementation (PCA) method (cf. Johnsson and Varshavsky, 1994 and WO9529195).

In the Cyto Trap method, cytoplasmic protein/protein interaction induces oncogene RAS activation and growth of a temperature-sensitive cdc25 yeast mutant. Briefly, a bait protein is located in the membrane and proteins (library) are fused to the human SOS protein which is able to activate RAS. Interaction with the bait protein activates the RAS pathway and thereby confers on the yeast the ability of growing at 37° C. A major drawback of this strategy is that the screening is based on a single mutation in the cdc25 gene, and reversion of the genotype can give false positives.

Protein fragment complementation assays (PCAs) were first implemented in the Split-ubiquitin system. This method is based on the re-assembly of N-and C-terminal halves of ubiquitin (Nub and Cub) fused to interacting proteins. The reassembled “native” ubiquitin is recognised and cleaved by ubiquitin specific protease. A reporter protein is released when interaction occurs. Derivative methods have also been described. The common principle of these techniques is that the reporter protein itself is split into two fragments. They are fused to the potential interaction partners and the interaction of the proteins restores the function of the cleaved reporter. Reporter proteins used for this purpose are enzymes such as beta-galactosidase (Rossi et al, 1997 and WO9844350), beta-lactamase (Wehrman et al, 2002 and WO03058197), or reporter fluorescent proteins such as GFP or YFP (Remy, 2004 and WO2004092336). In these methods, the bait protein is genetically fused to another protein.

A major drawback of this system is that controls cannot be performed in order to ensure the functional integrity of this protein when fused to the bait. For example, in the Yeast two hybrid method, when the bait is fused to a DNA binding domain or a transcription activation domain, it can disable the function of the DNA binding domain or of the transcription activation domain. In this case, even if interaction occurs, the reporter gene is not transcribed.

Another method was described in US2005048477. US2005048477 discloses methods for cytoplasmic detection of protein-protein interactions, nuclear export/localization sequences, and galactose-independent inducible Gal4p-mediated gene expression through the utilization of GAL regulatory factor, Gal80p, and the yeast galactose regulon.

### SUMMARY OF THE INVENTION

The present invention provides a versatile and sensitive method for detecting and/or selecting a peptide or polypeptide which binds to a bait peptide or polypeptide within the cytoplasm of a host cell.

The invention is based on the aggregating property of certain protein domains called aggregation domains. When a peptide or a polypeptide is fused to such an aggregation domain, the chimeric polypeptide obtained when expressed in a suitable host cell where the aggregation domain can form aggregates will form part of such an aggregate. A polypeptide interacting with such a chimeric polypeptide which forms part of a protein aggregate will be entrapped within said aggregate.

The present invention is based on the use of two distinct chimeric polypeptides. The first chimeric polypeptide contains an aggregation domain fused to a polypeptide A and the second contains a phenotype-associated functional domain fused to a second polypeptide B. When these chimeric polypeptides are co-expressed within a host cell allowing aggregation of the aggregation domain, the phenotype of said host cell depends on the entrapment of the phenotype-associated functional domain, which only occurs when A and B interact with each other.

### DETAILED DESCRIPTION OF THE INVENTION

The present invention provides a method for detecting the intracellular interaction between two polypeptides or proteins A and B.

For this purpose, A is fused to an aggregation domain X, leading to the sequestration of the entire X-A fusion in aggregates of host cell allowing X aggregation.

B is fused to Y, a phenotype associated functional domain, to form the B—Y fusion. Neither B nor Y contains an aggregation domain allowing aggregation with X. When A and B interact, Y is sequestered in aggregates and not functional. Inversely, if A and B do not interact, Y is free and functional. As the function of Y is associated with a phenotype, the interacting status of A and B can be visualised.

The present invention provides a kit for selecting a peptide or polypeptide A which binds to a peptide or polypeptide B comprising:
a first expression vector comprising a nucleotide sequence encoding a first chimeric polypeptide of formula X-A, wherein A is a polypeptide or a peptide and X is a polypeptide or peptide which comprises an aggregation domain wherein said aggregation domain enables the aggregation in a host cell of said chimeric polypeptide; and
a second expression vector comprising a nucleotide sequence encoding a second chimeric polypeptide of formula B—Y, wherein B is a polypeptide or peptide and Y is a polypeptide or a peptide, comprising a phenotype-associated functional domain,

wherein when B—Y and X-A are both expressed in a host cell in which X-A is entrapped in aggregates, the phenotype of said host cell depends on whether Y is entrapped or not in an aggregate, Y being entrapped only if A and B interact with each other.

Neither B nor Y contains an aggregation domain allowing aggregation with X, the phenotype of said host cell depends on whether B—Y is entrapped or not in an aggregate, B—Y being entrapped only if A and B interact with each other.

The two different components of the chimeric polypeptide of formula X-A or B—Y can be directly linked or linked via a spacer comprised of a peptide of 1 to 20 amino acids. X or B may be linked to the N-terminal or to the C-terminal amino-acid of A and Y respectively.

In a specific embodiment of the present invention, if the polypeptide A already comprises an aggregation domain, which enables aggregation in a host cell of said polypeptide A, the presence of a further aggregation domain may not be needed. In this specific case, a suitable first expression vector may be an expression vector comprising a nucleotide sequence encoding a polypeptide A comprising an aggregation domain, which enables the aggregation in a host cell.

Typically A or B may be expression products issued from a cDNA library or from a library resulting from the construction of nucleotide sequences repertories, said nucleotide sequences being characterised in that said nucleotides differ by at least one base. The generation of the variant nucleotide sequences encoding A or B may be performed by site-directed mutagenesis, preferentially by random mutagenesis.

In a preferred embodiment, A or B is an immunoglobulin, or a member of the immunoglobulin super-family, or any fragment thereof. In this context, the term immunoglobulin includes members of the classes IgA, IgD, IgE, IgG, and IgM. The term immunoglobulin super-family refers to all proteins which share structural characteristics with the immunoglobulins, including, for example, the T-cell receptor, or any of the molecules CD2, CD4, CD8 etc. Also included are fragments which can be generated from these molecules, such as Fv (the two variable regions), single chain Fv (an Fv complex in which the component chains are joined by a linker molecule), Fab, F(ab′)₂ or an immunoglobulin domain, such as the constant fragment (Fc), the variable heavy chain domain (VH) or the variable light chain domain VL.

Typically a kit according to an embodiment of the present invention further comprises a host cell wherein when said first and second expression vectors are introduced into said host cell, said host cell expresses chimeric polypeptides X-A and B—Y and X-A forms part of a protein aggregate which is present in said host cell and the phenotype of said host cell depends if B—Y (Y) is entrapped or not in said aggregate.

Typically said host cell is a prokaryotic or an eukaryotic cell, preferably a yeast cell or a mammalian cell.

Typically the aggregation domain is naturally able to form aggregate within said host cell. Alternatively the skilled person may use existing protocols such as the ones developed for studying prions to transform a host cell in such a way that the host cell expresses polypeptides which promotes the formation of protein aggregates within the host cell.

It falls within the ability of the skilled person to select or engineer a polypeptide or a peptide X, comprising an aggregation domain, which enables the aggregation in a host cell of said chimeric polypeptide. The skilled person may for example select a polypeptide or a peptide X comprising an aggregation domain which is already present in said host cell. Aggregation domains within prion proteins are well known (see for review Chernoff, 2004). Furthermore it has been demonstrated that the ability of forming protein aggregate can be conferred to a protein by adding a very short amino acid stretch (see for example Ventura et al., 2004).

Typically X comprises an aggregation domain selected from the group consisting of the Sup35 amino-terminal domain, a domain which is rich in glutamine and asparagines, a domain consisting of 50 to 400 glutamine residues and an intracellular insoluble domain.

Intracellular insoluble domains form aggregate due to their insolubility.

In the case where X is the sup35 amino-terminal domain, the host cell should have pre-existing endogen sup35 or other prion protein aggregates allowing the induction of the X-A aggregation (cf. Derkatch et al, 2001 and Osherovich et al, 2001).

Typically X is an amyloidogenic polypeptide. Examples of amyloidogenic polypeptides are prion proteins (e.g. Sup35, New1 Ure2, PrP), huntingtin, alpha-syneclein, beta-amyloid peptide or fragments and derivatives thereof having aggregating properties.

By fragment and derivative thereof it is meant a polypeptide, which is obtainable by substitution, deletion and/or addition of one or several amino acids in the original amino acid sequence.

It falls within the ability of the skilled person to select a polypeptide or a peptide Y comprising a phenotype-associated functional domain, wherein when B—Y is expressed in a host cell the phenotype of said host cell depends if B—Y is entrapped or not in a protein aggregate, B—Y being entrapped if A and B interact with each other.

Typically Y is a polypeptide working with poorly diffusible or specifically located substrates (e.g. polyosomes in the case of Sup35). The sequestration within a protein aggregate of such a polypeptide results in decreased function due to decreased access to a substrate. It falls within the ability of the skilled person to select or engineer a host cell in which the decreased function is associated with a modification of the phenotype of said host cell. For this purpose the skilled person may use one or more reporter proteins.

Typically Y may be selected from the group consisting of a translation termination factor, a transcription factor and an enzyme.

In one embodiment of the present invention, a kit is provided, which comprises:

a first expression vector comprising a nucleotide sequence encoding a first chimeric polypeptide of formula X-A as defined previously; and

a second expression vector comprising a cloning site which enables the introduction of a nucleotide sequence encoding a peptide or polypeptide B in such a way that the chimeric polypeptide of formula B—Y as defined previously is expressed in a host cell when said expression vector is introduced into said host cell.

In one embodiment of the present invention, a kit is provided, which comprises:

a first expression vector comprising a cloning site which enables the introduction of a nucleotide sequence encoding a peptide or polypeptide A in such a way that the chimeric polypeptide of formula X-A as defined previously is expressed in a host cell when said expression vector is introduced into said host cell; and

a second expression vector comprising a cloning site which enables the introduction of a nucleotide sequence encoding a peptide or polypeptide B in such a way that the chimeric polypeptide of formula B—Y as defined previously is expressed in a host cell when said expression vector is introduced into said host cell.

In one embodiment of the present invention, a kit is provided, which comprises:

a first expression vector comprising a cloning site which enables the introduction of a nucleotide sequence encoding a peptide or polypeptide A in such a way that the chimeric polypeptide of formula X-A as defined previously is expressed in a host cell when said expression vector is introduced into said host cell; and
a second expression vector comprising a nucleotide sequence encoding a second chimeric polypeptide of formula B—Y as defined previously.

In another embodiment of the present invention a kit is provided, which comprises:

a first expression vector comprising a nucleotide sequence encoding a first chimeric polypeptide of formula X-A as defined previously or a first expression vector comprising a cloning site which enables the introduction of a nucleotide sequence encoding a peptide or polypeptide A in such a way that the chimeric polypeptide of formula X-A is expressed in a host cell when said expression vector is introduced into said host cell; and

a library of second expression vectors each comprising a nucleotide sequence encoding a chimeric polypeptide of formula B—Y as defined previously, wherein B is a polypeptide or peptide which varies within the library.

In another embodiment of the present invention a kit is provided, which comprises:

a library of first expression vectors, each comprising a nucleotide sequence encoding a first chimeric polypeptide of formula X-A as defined previously, wherein A is a polypeptide or peptide which varies within the library; and

a second expression vector comprising a nucleotide sequence encoding a second chimeric polypeptide of formula B—Y as defined previously or a second expression vector comprising a cloning site which enables the introduction of a nucleotide sequence encoding a peptide or polypeptide B in such a way that the chimeric polypeptide of formula B—Y is expressed in a host cell when said expression vector is introduced into said host cell.

Typically the libraries result from the construction of nucleotide sequences repertories, nucleotide sequences characterised in that they are different by at least one change. The generation of the variant nucleotide sequences encoding A or B may be performed by site-directed mutagenesis, preferentially by random mutagenesis. Random mutagenesis can be performed by using a mutase, Pol beta for example (see WO0238756).

In another embodiment of the present invention a host cell is provided, which enables the expression of the first and the second expression vector as defined previously. Typically said host cell is a prokaryotic or an eukaryotic cell, preferably a yeast cell or a mammalian cell.

Typically the expression vectors are introduced into the host cell by standard co- or sequential transformation methods.

Alternatively, if the host cell is a yeast cell, mating may be used in order to improve the cotransformation efficiency.

In a further embodiment of the present invention, a kit is provided, which comprises:

a first haploid host yeast cell comprising the first expression vector as defined previously; and

a second haploid host yeast cell comprising the second expression vector as defined previously,

wherein said first and said second haploid yeast host cells are of opposite mating type.

In another embodiment of the present invention a kit is provided, which comprises:

a first haploid host yeast cell comprising the first expression vector as defined previously; and

a library of second haploid host yeast cells, each comprising the second expression vector encoding a chimeric polypeptide of formula B—Y as defined previously, wherein B is a polypeptide or peptide which varies within the library,

wherein said first and said second haploid host cells are of opposite mating type.

In another embodiment of the present invention a kit is provided, which comprises:

a library first haploid host yeast cell, each comprising the first expression vector as defined previously, wherein A is a polypeptide or peptide which varies within the library; and

a second haploid yeast host cells comprising the second expression vector encoding a chimeric polypeptide of formula B—Y as defined previously; wherein said first and said second haploid host yeast cells are of opposite mating type.

An example of suitable host yeast cell for carrying out the present invention when Y is a translational termination factor, is a host yeast cell containing a nonsense mutation in the ade1 gene or in another gene in which the nonsense mutation induces a phenotype switch.

In another embodiment of the present invention, a method for selecting a peptide or polypeptide B which binds to a peptide or polypeptide A is provided. A or B may be used as bait. Typically for performing this method the skilled person may use the kits and host cells defined previously.

In one embodiment of the present invention, a method for selecting a peptide or polypeptide B which binds to a bait peptide or polypeptide A is provided, which comprises the steps of:

a) generating a library of host cells, each expressing the two chimeric polypeptides X-A and B—Y as defined previously and wherein B is a polypeptide or peptide which varies within the library;

b) detecting a change of phenotype within the host cells of the library;

c) isolating from the host cells with a modified phenotype the expression vector encoding B—X;

d) determining the nucleotide sequence encoding B from the expression vector isolated in step c).

In an alternative embodiment of the present invention, a method for selecting a peptide or polypeptide A which binds to a bait peptide or polypeptide B is provided, which comprises the steps of:

a) generating a library of host cells, each expressing the two chimeric polypeptides X-A and B—Y as defined previously and wherein A is a polypeptide or peptide which varies within the library;

b) detecting a change of phenotype within the host cells of the library;

c) isolating from the host cells with a modified phenotype the expression vector encoding X-A;

d) determining the nucleotide sequence encoding A from the expression vector isolated in step c).

Once the nucleotide sequence of B or A is determined, B or A can be easily produced.

One of the advantages of these methods is the reduced number of false positive results. Easy control can be performed. The presence of X-A in an aggregate can easily be assessed and the effect of the presence of B on the phenotype-associated functional domain also.

Typically in the method for selecting a peptide or polypeptide B which binds to a bait peptide or polypeptide A, in order to generate a library of host cells, each expressing the two chimeric polypeptides X-A and B—Y, the following steps may be followed:

i) generating a library of host cells, expressing the chimeric polypeptides B—Y, wherein B is a polypeptide or peptide which varies within the library;

ii) selecting the host cells having the phenotype-associated with Y;

iii) transforming the host cells selected in such a way that they express the chimeric polypeptides X-A as defined previously.

Typically in the method for selecting a peptide or polypeptide A which binds to a bait peptide or polypeptide B, the following control may be performed:

B—Y is expressed in a host cell in order to check if when Y fused to B, the phenotype-associated functional domain of Y is still active. Typically the method comprises the following steps:
i) generating host cell, expressing the chimeric polypeptide B—Y, wherein B is a bait peptide.
ii) Selecting host cells having the phenotype associated with Y.
iii) Transforming the selected host cells in such a way that a library of host cells is generated, each expressing the two chimeric polypeptides X-A and B—Y wherein A is a polypeptide or peptide which varies within the library.

In contrast with Yeast two hybrid method, the control performed in step ii), enables the confirmation before screening that the functional domain Y is able to ensure its function in a fusion context.

In another embodiment of the present invention a method for screening compounds interfering with the interaction of two polypeptides A and B is provided, which comprises the steps of:

a) adding the compound to be screened to a host cell expressing the two chimeric polypeptides X-A and B—Y as defined previously;

b) detecting a change of phenotype of said host cell.

Typically if the compounds to be screened do not easily cross the cytoplasmic membrane of the host cell, the skilled person may use existing protocols such as cell permeabilization techniques in order to facilitate the entry into the cell of the compounds.

In the following, the invention will be illustrated by means of the following examples as well as the figures.

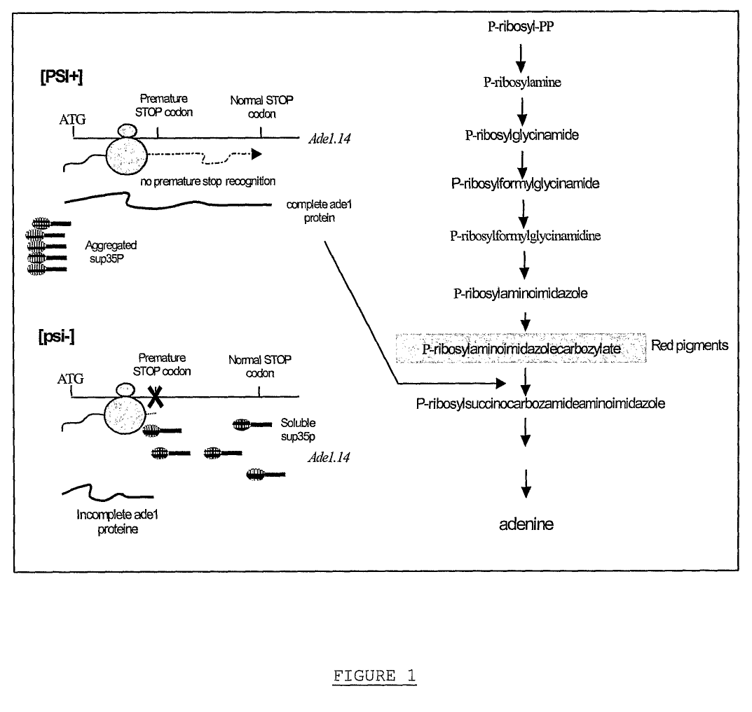

FIG. 1 shows the general mechanism which occurs in yeast 74D694 strain explaining the [PSI+] and [psi−] phenotypes.

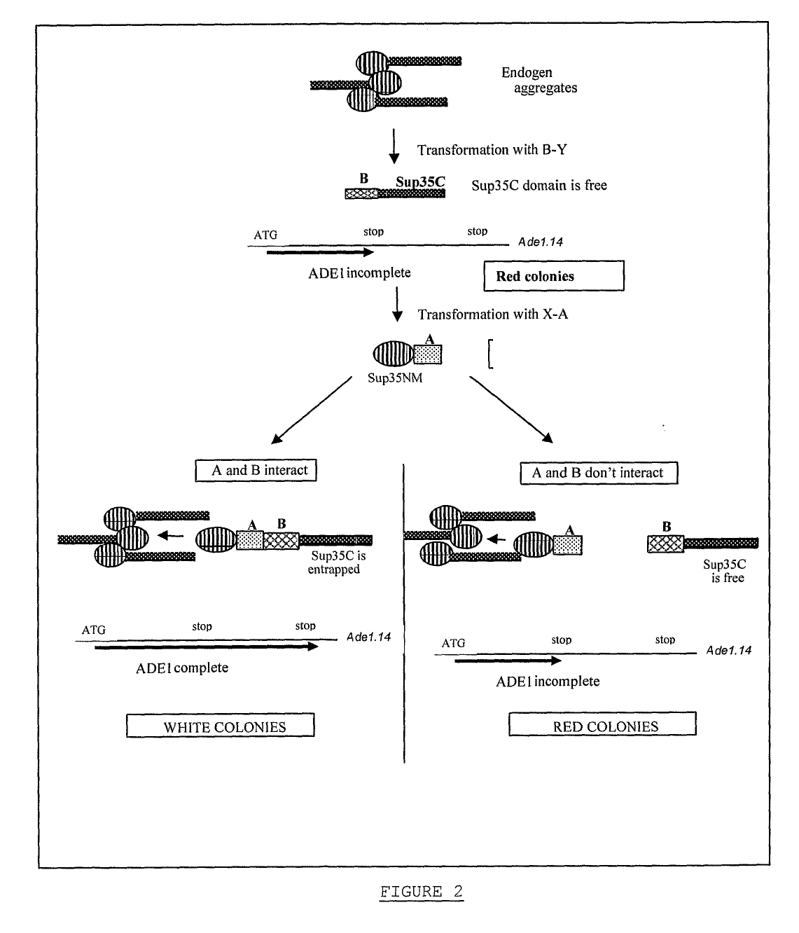

FIG. 2 is a schematic representation of the molecular events occurring in a yeast cell where endogen sup35 aggregates are present, leading to a change of phenotype. The method is exemplified with the protein sup35 (abbreviated as sup35 or sup35p). In the present case X is sup35NM and Y is sup35C.

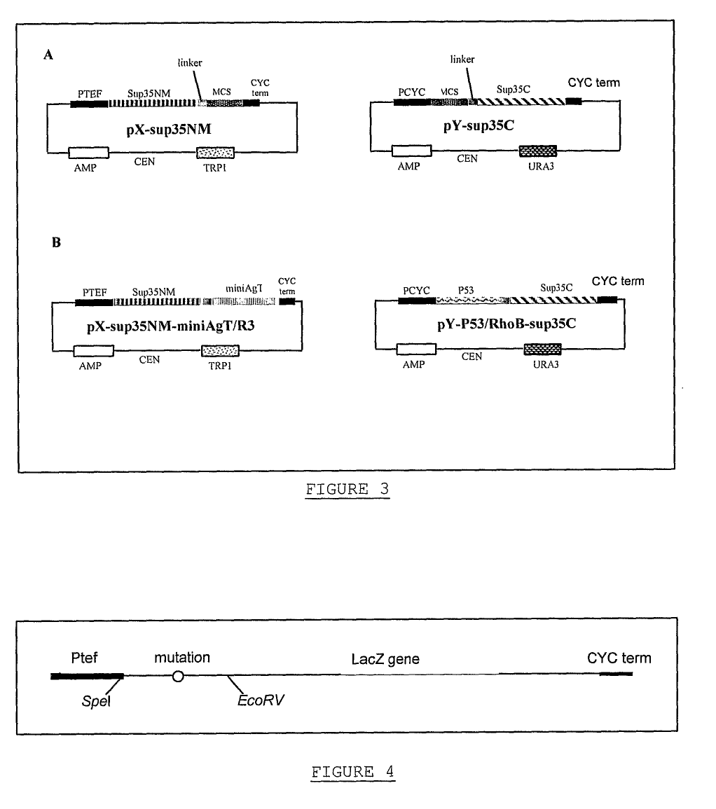

FIG. 3 illustrate different constructs that allow expression of different fusion proteins used in the examples.

FIG. 4 is a schematic representation of the mutated lacZ gene. The mutation is represented (circle) and the SpeI and EcoRV restriction sites used to construct the yeast expression plasmid pTEF-lacZ-mut are also represented.

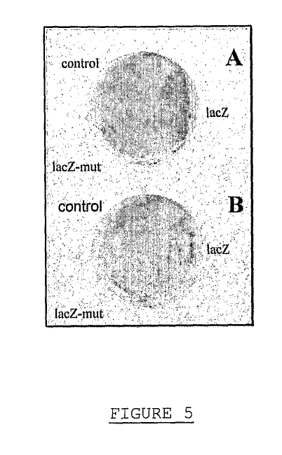

FIG. 5 are pictures of petri dishes with colonies transformed with lacZ or lacZmut gene platted on X-gal containing medium (A) or X-gal+GuHCl containing medium (B).

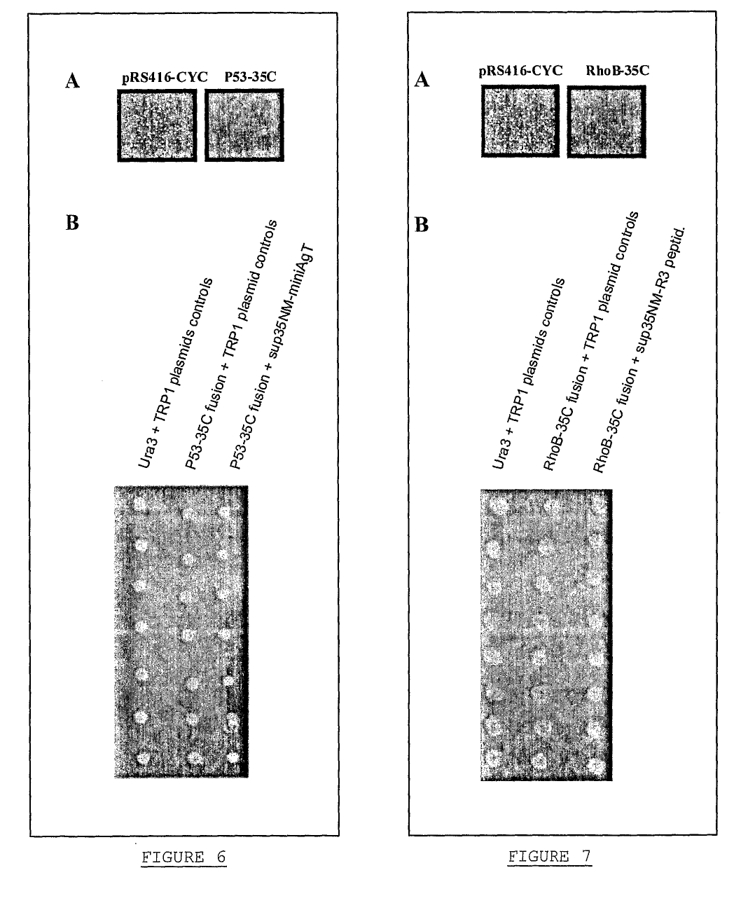

FIGS. 6 and 7 are pictures of colonies transformed with different constructs.

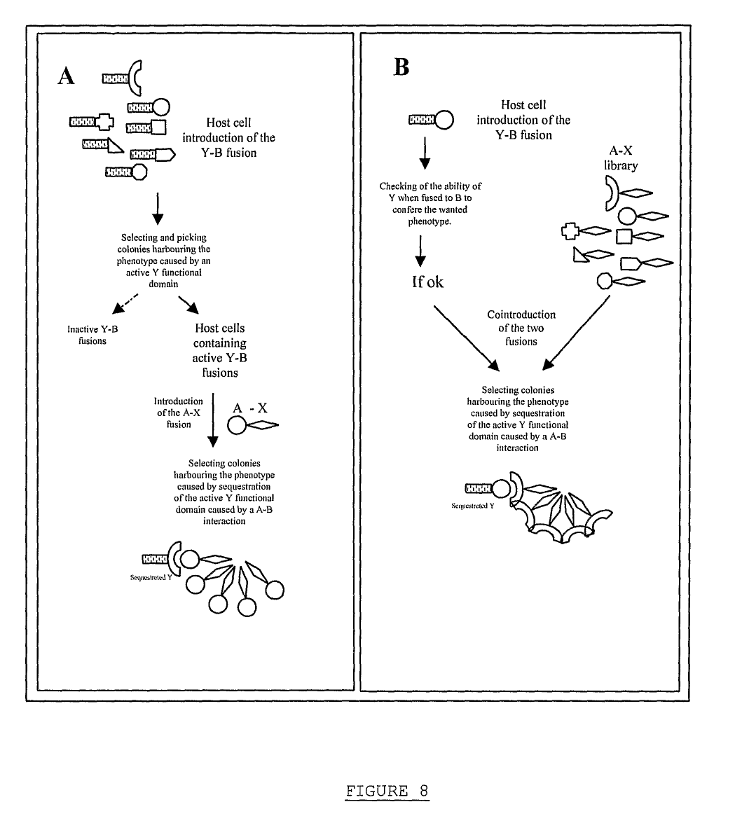

FIGS. 8A and 8B are schematic representations of examples of suitable strategies for screening libraries to find a bait interacting polypeptide or peptide. In FIG. 8A, A is the bait polypeptide or peptide and B varies within the library. In FIG. 8B, B is the bait polypeptide or peptide and A varies within the library.

FIG. 9 shows the study of the lacZ-mut1 expression in presence of different concentrations of GuHCl. After overnight culture, cells are lysed and beta-galactosidase activity is measured at several times.

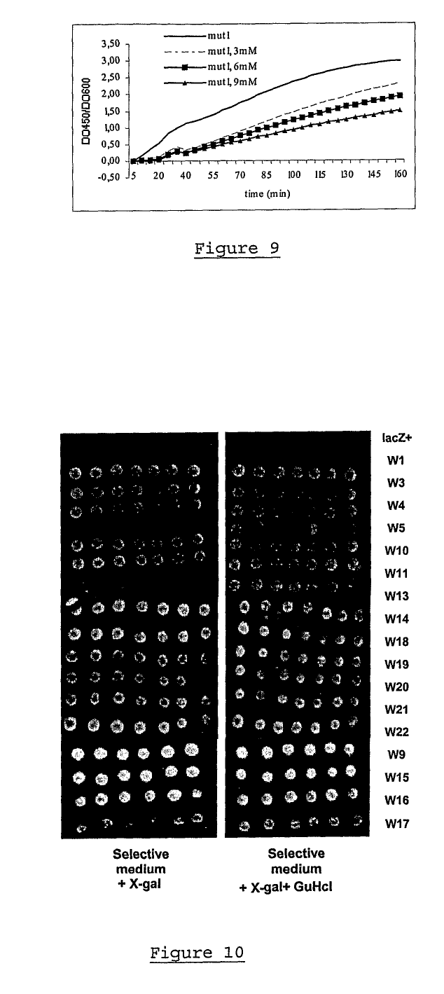

FIG. 10 is a picture of spots obtained with colonies of yeast transformed with the mutants in selective medium containing X-gal +/−GuHCl.

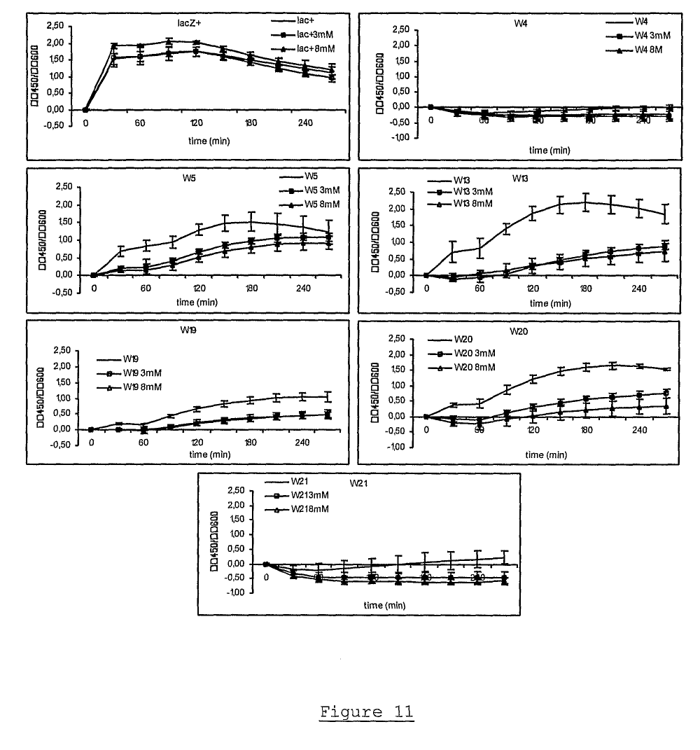

FIG. 11 shows the study and comparison of the translation of the different mutants in presence or not of GuHCl.

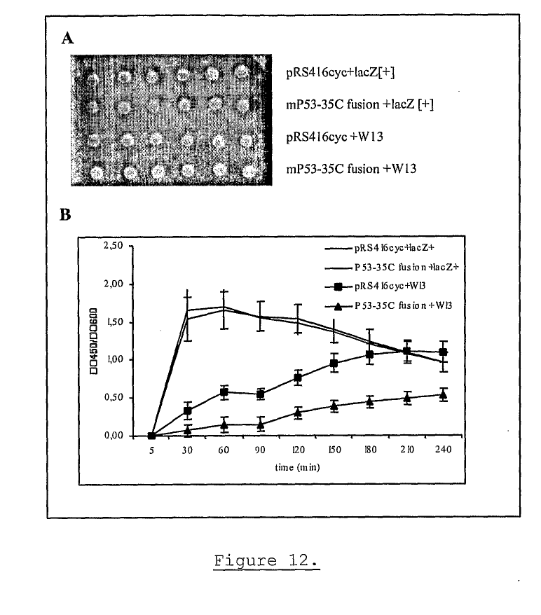

FIG. 12 shows the study of the effect of free sup35C fused to a bait (P53) on the translation of wild type LacZ and W13 mutant.

### EXAMPLES

In the following description, all molecular biology experiments are performed according to standard protocol (Sambrook et al, 1989).

1—Vectors and Cell Engineering

Yeast 74D694 strong [PSI+] strain (Mata, adel.14, trp-289, his3Δ200, ura3-52, leu2-3,112) was used for all the studies.

a. Cloning of sup35 Gene

Primers sup35a: 5′-CGGGATCCGCATGTCGGATTCAAACCAAG-3′ (SEQ ID NO:1) and sup35b: 5′-CGGAATTCGCTTACTCGGCAATTTTAACAATTTTACC-3′ (SEQ ID NO:2) containing respectively BamHI and EcoRI restriction sites were used to amplify sup35 gene (2055 pb) from total genomic DNA extract from S. cerevisiae EYB100 strain. Sup35 was cloned on the pMG71 plasmid between BamHI and EcoRI restriction sites to obtain the pMG-sup35.

b. Construction of the Vector pY-sup35C

The sequence encoding the C terminal domain of sup35 (sup35C) (aa 255-685) was cloned in frame with a linker in a pY plasmid containing the weak PCYC promoter, the URA3 auxotrophic marker and the low copy CEN yeast replication origin. The vector pY-sup35C was constructed from pRS416-PCYC (Mumberg et al, 1995) into which the C terminal domain of sup35 (sup35C) was cloned. For this purpose, PCR amplification with primers fussup35g: 5′-TATACCAAGCTTTTTGGTGGTAAAGATCACG-3′ (SEQ ID NO:3) and fus sup35h: 5′-TATACCGCTCGAGCGGTTACTCGGCAATTTTAAC-3′ (SEQ ID NO:4) containing respectively HindIII and XhoI restriction sites was performed using pMG-sup35 as template. This PCR fragment was cloned into the pRS416-PCYC plasmid between HindIII and XhoI restriction sites. Fragment containing the linker was PCR amplified from the plasmid p03 using primers fus sup35e: 5′-TATAGGGAATTCAGTGGTGGTGGTTCTGG-3′ (SEQ ID NO:5) and fus sup35f: 5′-TATACCCAAGCTTGCGCTTATCGTCATCGTC-3′ (SEQ ID NO:6) containing respectively EcoRI and HindIII restriction sites. This fragment was cloned between EcoRI and HindIII restriction sites into pRS416-PCYC-sup35C in frame with sup35C to obtain the pY-sup35C.

c. Construction of the Vector pX-sup35NM

The sequence encoding the N-terminal domain of sup35 (sup35NM) (aa 1-253) was cloned in frame with a linker into the pX plasmid containing a strong PTEF promoter, the TRP-1 auxotrophic marker and the high copy 2 μ yeast replication origin. First sup35NM was PCR amplified using pMG-sup35 as template with primers fus sup35a: 5′-TATAGCTCTAGAGCATGTCGGATTCAAACCAAG-3′ (SEQ ID NO:7) and fus sup35b: 5′-TATACGACTAGTATCGTTAACAACTTCGTC-3′ (SEQ ID NO:8) containing respectively XbaI and SpeI restriction sites. sup35NM was cloned into the PRS416-PTEF yeast plasmid to obtain pTEF-SUP35NM. Fragment containing linker was PCR amplified using primers fus sup35c: 5′-TATACGGACTAGTGGTGGTGGTGGTTC-3′ (SEQ ID NO:9) and fus sup35d: 5′-TATAGGGATCCGCGCTTATCGTCATCGTCGTAC-3′ (SEQ ID NO:10) containing respectively SpeI and BamHI restriction sites with p03 as template. This linker was then cloned in frame with sup35NM to obtain pTEFsup35NM linker. To replace the URA3 auxotrophic marker by TRP1 one, SwaI/EcoRI fragment of a plasmid previously construct in the laboratory, the MCS of pMGl-mscpGAL was inserted in place of the SwaI/EcoRI fragment of the PTEFsup35NM linker.

2—Development of a Reporter System.

In addition of the white/red phenotype a second reporter system was developed to facilitate the detection of colonies containing interacting proteins. A mutated version of the lacZ gene was constructed to allow a blue/white phenotype and positive selection of candidates by flow cytometry.

a. Example of Construction of a Mutated Reporter Gene: the lacZ Gene.

A substitution was made leading to a stop codon in the lacZ ORF. This mutation was performed in order to render lacZ translation dependent on the sup35 prion protein status. With such mutation, when sup35 or sup35C is free, sup35 or sup35C stops the lacZ translation at the internal stop codon. LacZ substrates are not hydrolysed. In contrast, when sup35 or sup35C is entrapped in aggregates, full translation occurs, lacZ mRNA is fully translated and the enzyme produced allows the hydrolysis of X-gal or derivative thereof. The lacZ mutated gene was constructed by overlap PCR. Primers couple: lacZdel1: 5′-GAAAGACTAGTCCAGTGTGGTGGAATTCTGC-3′ (SEQ ID NO:11)/lacZdel1′: 5′-TTTCACCCTGTCATAAAGAAACTGTTACCCGTA-3′ (SEQ ID NO:12) and lacZdel2: 5′-TACGGGTAACAGTTTCTTTATGACAGGGTGAAAC-3′ (SEQ ID NO:13)/lacZdel2′: 5′-CAGCAGGATATCCTGCACCATCGTCTG-3′ (SEQ ID NO:14) were used to perform a first PCR step. lacZdel1′ and lacZdel2 overlapping primers contain the mutation which introduce a Stop codon in the codon 257 of lacZ ORF. After this first step, PCR products were used as template for a third PCR step with primers lacZdel1 and lacZdel2′ containing SpeI and EcoRV restriction sites to clone the mutated lacZ fragment (cf. FIG. 4). The SpeI and EcoRV digested PCR fragment was introduced in order to replace the wild type fragment of the lacZ gene in the pTracer-lacZ plasmid. The entire lacZ gene containing the mutation (lacZmut) was then cloned into the pRS416-TEF yeast expression plasmid.

b. lacZ-mut Translation is Function of the sup35 Aggregated/Soluble Status.

To test the functionality of the mutated lacZ gene in this system, the 74D694 strain was transformed with a control empty plasmid (pRS416-TEF), a plasmid coding the wild type lacZ gene or a plasmid coding the lacZ-mut gene. Transformation products were platted in medium lacking uracile and containing X-gal with or without guanidine hydrochloride (GuHCl). GuHCl was added as control because GuHCl is known to dissociate sup35 aggregates. When GuHCl is added to the medium, sup35 is soluble and stops translation at the premature stop codons.

Results are shown in FIG. 5. When yeast cells transformed with the wild type lacZ gene are platted in Xgal containing medium or X-gal+ GuHCl containing medium, colonies are blue. The gene translation is not stopped at the premature stop codon when sup35 forms part of a protein aggregate. When yeast is transformed with the lacZ-mut gene, colonies are blue when platting in medium containing X-gal and are red in medium containing Xgal+ GuHCl. This phenotype change is due to the solubilization of sup35 by GuHCl which can stop translation in premature stop codon (lacZ-mut and ade1.14)

A 96 well microplate quantitative study was then performed. For this purpose the Yeast β-galactosidase assay kit from Pierce was used and measures were normalised with the OD at 600 nm of each corresponding well.

Results are shown in FIG. 9.

GuHCl concentration has an effect on the lacZ mut1 mutant translation.

These results show that this approach is useful to isolate a lacZ mutant which can be used as a reporter system to detect colonies wherein sup 35C is sequestered in aggregates.

c. Construction of Several lacZ Mutants.

As previously described, 15 other mutants were constructed with overlap PCR. (For primers details see table 1). PCR Fragments containing mutations were introduced instead of wild type sequence in PTEF-LEU2-LacZ.

TABLE 1

| co- | | | | | |
|---|---|---|---|---|---|
| don | mutants | overlap primers | | external primers | |
| 149 | W1 | seq ID N^{o}21 | seq ID N^{o}22 | seq ID N^{o}51 | seq ID N^{o}52 |
| 187 | W3 | seq ID N^{o}23 | seq ID N^{o}24 | seq ID N^{o}51 | seq ID N^{o}52 |
| 199 | W4 | seq ID N^{o}25 | seq ID N^{o}26 | seq ID N^{o}51 | seq ID N^{o}52 |
| 257 | W5 | seq ID N^{o}27 | seq ID N^{o}28 | seq ID N^{o}11 | seq ID N^{o}53 |
| 452 | W9 | seq ID N^{o}29 | seq ID N^{o}30 | seq ID N^{o}54 | seq ID N^{o}40 |
| 470 | W10 | seq ID N^{o}31 | seq ID N^{o}32 | seq ID N^{o}54 | seq ID N^{o}40 |
| 514 | W11 | seq ID N^{o}33 | seq ID N^{o}34 | seq ID N^{o}54 | seq ID N^{o}40 |
| 549 | W13 | seq ID N^{o}35 | seq ID N^{o}36 | seq ID N^{o}54 | seq ID N^{o}40 |
| 566 | W15 | seq ID N^{o}37 | seq ID N^{o}38 | seq ID N^{o}54 | seq ID N^{o}40 |
| 650 | W16 | seq ID N^{o}39 | seq ID N^{o}40 | seq ID N^{o}55 | seq ID N^{o}56 |
| 690 | W17 | seq ID N^{o}41 | seq ID N^{o}42 | seq ID N^{o}55 | seq ID N^{o}56 |
| 713 | W19 | seq ID N^{o}43 | seq ID N^{o}44 | seq ID N^{o}55 | seq ID N^{o}56 |
| 716 | W20 | seq ID N^{o}45 | seq ID N^{o}46 | seq ID N^{o}55 | seq ID N^{o}56 |
| 752 | W21 | seq ID N^{o}47 | seq ID N^{o}48 | seq ID N^{o}55 | seq ID N^{o}56 |
| 765 | W22 | seq ID N^{o}49 | seq ID N^{o}50 | seq ID N^{o}55 | seq ID N^{o}56 |

As previously described, to test the functionality of the mutated lacZ gene in this system, the 74D694 strain was transformed with a control empty plasmid (pRS416-TEF), a plasmid coding the wild type lacZ gene or a plasmid coding the lacZ-mut gene. Transformation products were platted in medium lacking leucine and containing X-gal with or without guanidine hydrochloride (GuHCl). GuHCl was added as control because GuHCl is known to dissociate sup35 aggregates. When GuHCl is added to the medium, sup35 is soluble and stops translation at the premature stop codons.

Results are shown in FIG. 10.

Colonies of cells transformed with mutants W4, W5, W13, W19, W20 and W21 are blue in selective medium containing X-gal. The same colonies are less blue when spotted in selective medium containing X-gal and GuHCl.

These results show that the translation of these mutants is dependent on the aggregated state of sup35. We performed a quantitative study in order to determine the best mutant for the method according to the invention.

Results are shown in FIG. 11.

Translation of all mutants is dependent on the sequestered form of sup35 in aggregates. The W13 mutant gives the best results with good level of lacZ expression when sup35 is aggregated and lacZ expression decreases

We chose to test its translation sensitivity to the presence of our 253-35C fusion.

For this purpose, co-transformations of plasmid coding for the W13 mutant and plasmid coding for P53-35C fusion were performed in the 74D694 strain.

Colonies from co-transformation were spotted in selective medium containing X-gal. In parallel a quantitative study was performed.

Results are shown in FIG. 12.

The presence of free sup35C fused to P53 has no effect on translation of wild type lacZ gene, whereas the W13 mutant translation is sensitive to the presence of free sup35C.

The W13 mutant is a good candidate for highlighting interactions between two proteins A and B with a method according to the invention.

3. Study of the Intracellular Protein Interaction of p53 and Mini Ag-T

The plasmids pX-sup35NM and pY-sup35C (FIG. 3A) were used to clone interacting proteins in order to test the method disclosed in the present invention.

The first couple tested was the well known p53/SV40-AgT which are used as positive interaction control in yeast two hybrid (clontech) and in MagneGST™ pull down (promega).

a. Cloning of p53 Gene into the Vector pY-sup35C

p53 was cloned in frame with sup35C into the pY-sup35C vector to obtain the pY-P53-sup35C. pY-P53-sup35C was constructed by cloning P53 PCR fragment from pGBKT7/p53 obtained with primers p53_{—}35c_{—}5′: 5′-TATATAACTAGTATGCCTGTCACCGAGACCCCT-3′ (SEQ ID NO:15) and p53_{—}35c_{—}31: 5′-TATATAGAATTCGTCTGAGTCAGGCCCCACTTTCTT-3′ (SEQ ID NO:16) containing SpeI and EcoRI restriction sites respectively in SpeI/EcoRI restriction sites in the pY-sup35C.

b. Cloning of MiniAgTgene into the Vector pX-sup35NM

The 880 pb length fragment from SV40AgT containing the P53 binding site was cloned in frame with sup35NM in pX-sup35NM to obtain the pX-sup35NM-miniAgT (FIG. 3B). pX-sup35NM-miniAgT was constructed by cloning mini-AgT PCR fragment from pGADT7/T obtained with primers 35N_miniAgT_{—}5′: 5′-TATATAACTAGTATGTTAGCTAAAAAGCGGGTTGATAGCC-3′ (SEQ ID NO:17) and 35N_miniAgT_{—}3′: 5′-TATATAGAATTCTTAATCTAAAACTCCAATTCCCATAGCCAC-3′ (SEQ ID NO:18) containing BamHI and EcoRI restriction site respectively in BamHI/EcoRI in the pX-sup35NM.

c. Test of the Ability of P53-sup35C Fusion to Reverse [PSI+] Phenotype in 74D694 Strain.

The strain 74D694 was transformed with pY-P53-sup35C and cells were plated on minimal medium YNB supplemented with Complete Supplement Mixture (csm) lacking uracile. A control was performed with the pTEF empty plasmid (without the sup35C coding sequence) and containing the URA3 auxotrophic marker. After four days at 30° C., the phenotype of the colonies was observed (cf. FIG. 6A). Control colonies transformed with the empty plasmid have a [PSI+] white/pink colour-based phenotype. Colonies transformed with plasmid coding the P53-sup35C fusion have a [psi−] red colour-based phenotype (cf. FIG. 6A).

These results indicate that sup35C fused with heterologous protein is able to stop translation at the premature STOP codon of adel-14 allele.

d. Test of the Ability of the Fusion sup35NM-miniAgT to Restore the [PSI+] Phenotype.

The 74D694 strain was cotransformed with the two plasmids: pY-P53-sup35C and pX-sup35NM-miniAgT.

Two controls were performed. The first one was a cotransformation of two empty plasmid harbouring TRP1 and URA3 auxotrophic markers to have a control of [PSI+] white/pink colour-based phenotype. The second control was cotransformation of pY-p53-sup35C with a plasmid harbouring TRP1 auxotrophic marker to dispose of the red phenotype control. The phenotypes of the colonies were observed after 4 days on minimal medium supplemented, with complete mixture lacking uracile and tryptophan. P53-sup35C fusion protein generated a [psi−] red colour-based phenotype when cotransformed with either an empty plasmid containing the TRP1 auxotrophic marker or a control plasmid encoding sup35NM fused to R3 peptide, a peptide which interacts with RhoB (cf. example 4), which does not interact with p53.

The colonies resulting from cotransformation with plasmids encoding P53-sup35C and sup35NM-miniAgT fusion proteins showed the [PSI+] white/pink colour-based phenotype (FIG. 6B). This result indicates that p53 interacts with miniAgT and consequently the ADE1 translation ending at the premature stop codon is prevented. Furthermore, the control colonies transformed with the two TRP1 and URA3 empty plasmids had [PSI+] white/pink colour-based phenotype. These results indicate that the system performs well for highlighting intracytoplasmic protein/protein interaction.

4. Study of the Intracellular Protein Interaction of RhoB and R3 Peptide.

The plasmids pX-sup35NM and pY-sup35C (FIG. 3A) were used to clone interacting proteins in order to test the method disclosed in the present invention. The second couple tested was RhoB/R3 peptides which have been described to interact in our laboratory.

a. Cloning of RhoB Gene into the Vector pY-sup35C

RhoB cloning sequence was cloned in frame with sup35C in the pY-sup35C vector to obtain the pY-RHOB-sup35C. This vector was constructed by cloning RhoB PCR fragment obtained with primers RhoB_{—}35c_{—}5′: 5′-TATAGGGATCCATGGCTTACCCATACGATG-3′ (SEQ ID NO:19) and RhoB_{—}35c_{—}3′: 5′-TATAGGGAATTCTAGCACCTTGCAGCAGTTG-3′ (SEQ ID NO:20). Primers contain SpeI and EcoRI restriction sites respectively to clone the RhoB containing fragment between SpeI/EcoRI restriction sites of the pY-sup35C (cf. FIG. 3B).

b. Cloning of R3 Gene into the Vector pX-sup35NM.

The 248pb BamHI/EcoRI length fragment from pMG-R3 containing R3 peptide coding sequence was cloned in frame with sup35NM in pX-sup35NM to obtain the pX-sup35NM-R3 (cf. FIG. 3B)

c. Test of the Ability of RHOB-sup35C Fusion to Reverse [PSI+] Phenotype in 74D694 Strain.

The strain 74D694 was transformed with pY-RHOB-sup35C and cells were plated on minimal medium YNB supplemented with csm (Complete Supplement Mixture) lacking uracile. A control was performed with the pTEF empty plasmid (without the sup35C encoding sequence) and containing the URA3 auxotrophic marker. After four days at 30° C. the phenotype of the colonies was observed (cf. FIG. 7A). Control colonies transformed with the empty plasmid have a [PSI+] white/pink phenotype. Colonies transformed with plasmid coding the RHOB-sup35C fusion have a [psi−] red phenotype. These results indicate that sup35C fused with heterologous protein is able to stop translation at the premature STOP codon of adel-14 allele.

d. Test of the Ability of the Fusion sup35NM-R3 to Restore the [PSI+] Phenotype.

The 74D694 strain was cotransformed with the two plasmids: pY-RHOB-sup35C and pX-sup35NM-R3.

Two controls were performed: The first one is cotransformation of two empty plasmid harbouring TRP1 and URA3 auxotrophic markers to have a control of [PSI+] white/pink color phenotype. The second control is a cotransformation of pY-RhoB-sup35C with a plasmid harbouring TRP1 auxotrophic marker to obtain the red phenotype control. The phenotypes of the colonies were observed after 4 days on minimal medium supplemented with complete amino acid mixture lacking uracile and tryptophan.

The fusion RHOB-sup35C protein confers a [psi−] red phenotype when cotransformed with either an empty plasmid containing the TRP1 auxotrophic marker or with a control plasmid coding a fusion between sup35NM and miniAgT, a protein that doesn't interact with RhoB. The colonies resulting from the cotransformation with plasmids coding the fusion proteins RHOB-sup35C and sup35NM-R3 showed the [PSI+] white/pink phenotype (FIG. 7B). This phenotype indicates that RhoB interacts with R3 and consequently the ADE1 translation ending at the premature stop codon is prevented. Furthermore, the control colonies transformed with the two TRP1 and URA3 empty plasmids had [PSI+] white/pink colour-based phenotype. These results indicate that the system performs well for highlighting intracytoplasmic protein/protein interaction.

### REFERENCES

Throughout this application, various references describe the state of the art to which this invention pertains. The disclosures of these references are hereby incorporated by reference into the present disclosure.
Broder Y C, Katz S, Aronheim A. The ras recruitment system, a novel approach to the study of protein-protein interactions. Curr Biol. 1998 Oct. 8;8(20):1121-4.
Chernoff, Yury O. Amyloidogenic domains, prions and structural inheritance: rudiments of early life or recent acquisition? Current Opinion in Chemical Biology, Volume 8, Issue 6, December 2004, Pages 665-671
Derkatch, I; Bradley, E; Hong, J and Liebman S. Prions affect the appearance of other prions: the story of [PIN+]. Cell, vol 106, Jul. 27, 2001, pages 171-182.
Fields, S; Song, O. A novel genetic system to detect protein-protein interactions. Nature, Volume 340, Issue 6230, Jul. 20, 1989, Pages 245-246
Johnsson, N; Varshavsky, A. Split ubiquitin as a sensor of protein interactions in vivo. Proceedings Of The National Academy of Sciences of The United States of America, Volume 91, Issue 22, Oct. 25, 1994, Pages 10340-10344.
Mumberg Dominik, Muller Rolf and Funk Martin. Yeast vectors for the controlled expression of heterologous proteins in different genetic backgrounds•Gene, Volume 156, Issue 1, 14 Apr. 1995, Pages 119-122.
Osherovich and Weissman. Multiple Gln/Asn-Rich prion domains confer sucsceptibility to induction of the yeast [PSI+] prion. Cell, volume 106, Jul. 27, 2001, pages 183-194.
Remy Ingrid and Michnick Stephen W. A cDNA library functional screening strategy based on fluorescent protein complementation assays to identify novel components of signaling pathways•Methods, Volume 32, Issue 4, April 2004, Pages 381-388
Rossi, F; Charlton, C A; Blau, H M. Monitoring protein-protein interactions in intact eukaryotic cells by beta-galactosidase complementation. Proceedings of The National Academy of Sciences Of The United States of America, Volume 94, Issue 16, Aug. 5, 1997, Pages 8405-8410
Sambrook, J; Fritsch, E. F; Maniatis, T. Molecular cloning, a laboratory manual. Cold spring harbor laboratory press, 1989
Ventura S, Zurdo J, Narayanan S, Parreno M, Mangues R, Reif B, Chiti F, Giannoni E, Dobson C M, Aviles F X, Serrano L. Short amino acid stretches can mediate amyloid formation in globular proteins:
   the Src homology 3 (SH3) case. Proc Natl Acad Sci USA. 2004 May 11; 101(19):7258-63.
Wehrman, Tom; Kleaveland, Benjamin; Her, Jeng-Horng; Balint, Robert F; Blau, Helen M. Protein-protein interactions monitored in mammalian cells via complementation of beta-lactamase enzyme fragments. Proceedings of The National Academy of Sciences of The United States Of America, Volume 99, Issue 6, Mar. 19, 2002, Pages 3469-3474

1. A kit comprising:
a first expression vector comprising a nucleotide sequence encoding a first chimeric polypeptide of formula X-A, wherein A is a polypeptide or a peptide and X is a polypeptide or peptide which comprises an aggregation domain wherein said aggregation domain enables the aggregation in a host cell of said chimeric polypeptide; and
a second expression vector comprising a nucleotide sequence encoding a second chimeric polypeptide of formula B—Y, wherein B is a polypeptide or peptide and Y is a polypeptide or a peptide, comprising a phenotype-associated functional domain,
wherein when B—Y and X-A are both expressed in a host cell in which X-A is entrapped in aggregates, the phenotype of said host cell depends on whether Y is entrapped or not in an aggregate, Y being entrapped only if A and B interact with each other.

2. The kit according to claim 1 wherein X comprises an aggregation domain, which enables the aggregation in a host cell of said chimeric polypeptide.

3. A kit comprising:
a first expression vector comprising a nucleotide sequence encoding a first polypeptide of formula A, wherein A is a polypeptide or a peptide which comprises an aggregation domain wherein said aggregation domain enables the aggregation in a host cell of said polypeptide A; and
a second expression vector comprising a nucleotide sequence encoding a chimeric polypeptide of formula B—Y, wherein B is a polypeptide or peptide and Y is a polypeptide or a peptide, comprising a phenotype-associated functional domain,
wherein when B—Y and A are both expressed in a host cell in which A is entrapped in aggregates, the phenotype of said host cell depends on whether Y is entrapped or not in an aggregate, Y being entrapped only if A and B interact with each other.

4. The kit according to claim 1 further comprising a host cell wherein when said first and second expression vectors are introduced into said host cell, said host cell expresses chimeric polypeptides X-A and B—Y, X-A forms part of an aggregate which is present in said host cell and the phenotype of said host cell depends if B—Y is entrapped or not in said aggregate.

5. The kit according to claim 4 wherein said host cell is a yeast cell or a mammalian cell.

6. The kit according to claim 1 wherein X comprises an aggregation domain selected from the group consisting of the Sup35 amino-terminal domain, a domain which is rich in glutamine and asparagines, a domain consisting of 50 to 400 glutamine residues and an intracellular insoluble domain.

7. The kit according to claim 1 wherein X is an amyloidogenic polypeptide selected from the group consisting of a prion protein, Sup35, New1, Ure2, PrP, huntingtin, alpha-syneclein and beta-amyloid peptide, fragments and derivatives thereof having aggregating properties.

8. The kit according to claim 1 wherein Y is selected from the group consisting of a translation termination factor, a transcription factor and an enzyme.

9. The kit according to claim 1 wherein A or B is an immunoglobulin, or a member of the immunoglobulin super-family, or any fragment thereof.

10. A kit comprising:
a first expression vector comprising a nucleotide sequence encoding a first chimeric polypeptide of formula X-A as defined in claim 1; and
a second expression vector comprising a cloning site which enables the introduction of a nucleotide sequence encoding a peptide or polypeptide B in such a way that the chimeric polypeptide of formula B—Y as previously defined is expressed in a host cell when said expression vector is introduced into said host cell.

11. A kit according comprising:
a first expression vector comprising a cloning site which enables the introduction of a nucleotide sequence encoding a peptide or polypeptide A in such a way that the chimeric polypeptide of formula X-A as defined in claim 1 is expressed in a host cell when said expression vector is introduced into said host cell; and
a second expression vector comprising a cloning site which enables the introduction of a nucleotide sequence encoding a peptide or polypeptide B in such a way that the chimeric polypeptide of formula B—Y as previously defined is expressed in a host cell when said expression vector is introduced into said host cell.

12. A kit according comprising:
a first expression vector comprising a cloning site which enables the introduction of a nucleotide sequence encoding a peptide or polypeptide A in such a way that the chimeric polypeptide of formula X-A as defined in claim 1 is expressed in a host cell when said expression vector is introduced into said host cell; and
a second expression vector comprising a nucleotide sequence encoding a second chimeric polypeptide of formula B—Y as previously defined.

13. A kit according to claim 1 comprising:
a first expression vector comprising a nucleotide sequence encoding a first chimeric polypeptide of formula X-A as previously defined or a first expression vector comprising a cloning site which enables the introduction of a nucleotide sequence encoding a peptide or polypeptide A in such a way that the chimeric polypeptide of formula X-A as previously defined is expressed in a host cell when said expression vector is introduced into said host cell; and
a library of second expression vectors each comprising a nucleotide sequence encoding a chimeric polypeptide of formula B—Y as previously defined, wherein B is a polypeptide or peptide which varies within the library.

14. A kit according to claim 1 comprising:
a library of first expression vectors each comprising a nucleotide sequence encoding a first chimeric polypeptide of formula X-A as previously defined, wherein A is a polypeptide or peptide which varies within the library; and
a second expression vector comprising a nucleotide sequence encoding a second chimeric polypeptide of formula B—Y as previously defined or a second expression vector comprising a cloning site which enables the introduction of a nucleotide sequence encoding a peptide or polypeptide B in such a way that the chimeric polypeptide of formula B—Y as previously defined is expressed in a host cell when said expression vector is introduced into said host cell.

15. A host cell comprising the first and the second expression vector as defined in claim 1.

16. The host cell according to claim 15 wherein said host cell is a yeast cell or a mammalian cell.

17. A kit comprising:
a first haploid host yeast cell comprising the first expression vector as defined in claim 1; and
a second haploid host yeast cell comprising the second expression vector as previously defined,
wherein said first and said second haploid host yeast cells are of opposite mating type.

18. A kit according to claim 17 comprising:
a first haploid host yeast cell comprising the first expression vector as previously defined; and
a library of second haploid yeast host cells, each comprising the second expression vector encoding a chimeric polypeptide of formula B—Y as previously defined, wherein B is a polypeptide or peptide which varies within the library;
wherein said first and said second haploid host yeast cells are of opposite mating type.

19. A kit according to claim 17 comprising:
a library first haploid host yeast cell, each comprising the first expression vector as previously defined, wherein A is a polypeptide or peptide which varies within the library; and
a second haploid yeast host cells comprising the second expression vector encoding a chimeric polypeptide of formula B—Y as previously defined; wherein said first and said second haploid host yeast cells are of opposite mating type.

20. A method for selecting a peptide or polypeptide B which binds to a bait peptide or polypeptide A comprising the steps of:
a) generating a library of host cells, each expressing the two chimeric polypeptides X-A and B—Y as previously defined and wherein B is a polypeptide or peptide which varies within the library;
b) detecting a change of phenotype within the host cells of the library;
c) isolating from the host cells with a modified phenotype the expression vector encoding B—Y;
d) determining the nucleotide sequence encoding B from the expression vector isolated in step c).

21. A method for producing a peptide or polypeptide B which binds to a bait polypeptide or peptide A comprising the steps of:
a) selecting the peptide or polypeptide B by performing the method of claim 20; and
b) producing B.

22. A method for selecting a peptide or polypeptide A which binds to a bait peptide or polypeptide B comprising the steps of:
a) generating a library of host cells, each expressing the two chimeric polypeptides X-A and B—Y as defined in claim 1 and wherein A is a polypeptide or peptide which varies within the library;
b) detecting a change of phenotype within the host cells of the library;
c) isolating from the host cells with a modified phenotype the expression vector encoding X-A;
d) determining the nucleotide sequence encoding A from the expression vector isolated in step c).

23. A method for producing a peptide or polypeptide A which binds to a bait polypeptide or peptide B comprising the steps of:
a) selecting the peptide or polypeptide A by performing the method of claim 22; and
b) producing A.

24. A method for screening compounds interfering with the interaction of two polypeptides A and B comprising the steps of:
a) adding the compound to be screened to a host cell as defined in claim 15 expressing the two chimeric polypeptides X-A and B—Y;
b) detecting a change of phenotype of said host cell.


SEQUENCE LISTING (56 sequences)

SEQ ID NO: 1 - DNA, 29 bp, Artificial
  Description of Artificial Sequence Synthetic primer
cgggatccgcatgtcggattcaaaccaag

SEQ ID NO: 2 - DNA, 37 bp, Artificial
  Description of Artificial Sequence Synthetic primer
cggaattcgcttactcggcaattttaacaattttacc

SEQ ID NO: 3 - DNA, 31 bp, Artificial
  Description of Artificial Sequence Synthetic primer
tataccaagctttttggtggtaaagatcacg

SEQ ID NO: 4 - DNA, 34 bp, Artificial
  Description of Artificial Sequence Synthetic primer
tataccgctcgagcggttactcggcaattttaac

SEQ ID NO: 5 - DNA, 29 bp, Artificial
  Description of Artificial Sequence Synthetic primer
tatagggaattcagtggtggtggttctgg

SEQ ID NO: 6 - DNA, 31 bp, Artificial
  Description of Artificial Sequence Synthetic primer
tatacccaagcttgcgcttatcgtcatcgtc

SEQ ID NO: 7 - DNA, 33 bp, Artificial
  Description of Artificial Sequence Synthetic primer
tatagctctagagcatgtcggattcaaaccaag

SEQ ID NO: 8 - DNA, 30 bp, Artificial
  Description of Artificial Sequence Synthetic primer
tatacgactagtatcgttaacaacttcgtc

SEQ ID NO: 9 - DNA, 27 bp, Artificial
  Description of Artificial Sequence Synthetic primer
tatacggactagtggtggtggtggttc

SEQ ID NO: 10 - DNA, 33 bp, Artificial
  Description of Artificial Sequence Synthetic primer
tatagggatccgcgcttatcgtcatcgtcgtac

SEQ ID NO: 11 - DNA, 31 bp, Artificial
  Description of Artificial Sequence Synthetic primer
gaaagactagtccagtgtggtggaattctgc

SEQ ID NO: 12 - DNA, 33 bp, Artificial
  Description of Artificial Sequence Synthetic primer
tttcaccctgtcataaagaaactgttacccgta

SEQ ID NO: 13 - DNA, 34 bp, Artificial
  Description of Artificial Sequence Synthetic primer
tacgggtaacagtttctttatgacagggtgaaac

SEQ ID NO: 14 - DNA, 27 bp, Artificial
  Description of Artificial Sequence Synthetic primer
cagcaggatatcctgcaccatcgtctg

SEQ ID NO: 15 - DNA, 33 bp, Artificial
  Description of Artificial Sequence Synthetic primer
tatataactagtatgcctgtcaccgagacccct

SEQ ID NO: 16 - DNA, 36 bp, Artificial
  Description of Artificial Sequence Synthetic primer
tatatagaattcgtctgagtcaggccccactttctt

SEQ ID NO: 17 - DNA, 40 bp, Artificial
  Description of Artificial Sequence Synthetic primer
tatataactagtatgttagctaaaaagcgggttgatagcc

SEQ ID NO: 18 - DNA, 42 bp, Artificial
  Description of Artificial Sequence Synthetic primer
tatatagaattcttaatctaaaactccaattcccatagccac

SEQ ID NO: 19 - DNA, 30 bp, Artificial
  Description of Artificial Sequence Synthetic primer
tatagggatccatggcttacccatacgatg

SEQ ID NO: 20 - DNA, 31 bp, Artificial
  Description of Artificial Sequence Synthetic primer
tatagggaattctagcaccttgcagcagttg

SEQ ID NO: 21 - DNA, 20 bp, Artificial
  Description of Artificial Sequence Synthetic primer
tttcatctgtgatgcaacgg

SEQ ID NO: 22 - DNA, 20 bp, Artificial
  Description of Artificial Sequence Synthetic primer
ccgttgcatcacagatgaaa

SEQ ID NO: 23 - DNA, 18 bp, Artificial
  Description of Artificial Sequence Synthetic primer
gctgcgttgaagtgacgg

SEQ ID NO: 24 - DNA, 18 bp, Artificial
  Description of Artificial Sequence Synthetic primer
ccgtcacttcaacgcagc

SEQ ID NO: 25 - DNA, 20 bp, Artificial
  Description of Artificial Sequence Synthetic primer
caggatatgtgacggatgag

SEQ ID NO: 26 - DNA, 20 bp, Artificial
  Description of Artificial Sequence Synthetic primer
ctcatccgtcacatatcctg

SEQ ID NO: 27 - DNA, 24 bp, Artificial
  Description of Artificial Sequence Synthetic primer
gtttctttatgacagggtgaaacg

SEQ ID NO: 28 - DNA, 24 bp, Artificial
  Description of Artificial Sequence Synthetic primer
cgtttcaccctgtcataaagaaac

SEQ ID NO: 29 - DNA, 19 bp, Artificial
  Description of Artificial Sequence Synthetic primer
cccagcgatcagatgatca

SEQ ID NO: 30 - DNA, 19 bp, Artificial
  Description of Artificial Sequence Synthetic primer
cccagcgatcagatgatca

SEQ ID NO: 31 - DNA, 20 bp, Artificial
  Description of Artificial Sequence Synthetic primer
gctgtatcgctgaatcaaat

SEQ ID NO: 32 - DNA, 20 bp, Artificial
  Description of Artificial Sequence Synthetic primer
atttgattcagcgatacagc

SEQ ID NO: 33 - DNA, 19 bp, Artificial
  Description of Artificial Sequence Synthetic primer
gatggatcatttcggcaca

SEQ ID NO: 34 - DNA, 19 bp, Artificial
  Description of Artificial Sequence Synthetic primer
gatggatcatttcggcaca

SEQ ID NO: 35 - DNA, 20 bp, Artificial
  Description of Artificial Sequence Synthetic primer
gctaaatactgacaggcgtt

SEQ ID NO: 36 - DNA, 20 bp, Artificial
  Description of Artificial Sequence Synthetic primer
aacgcctgtcagtatttagc

SEQ ID NO: 37 - DNA, 19 bp, Artificial
  Description of Artificial Sequence Synthetic primer
cgtctgggactgagtggat

SEQ ID NO: 38 - DNA, 19 bp, Artificial
  Description of Artificial Sequence Synthetic primer
atccactcagtcccagacg

SEQ ID NO: 39 - DNA, 18 bp, Artificial
  Description of Artificial Sequence Synthetic primer
gcactgaatggtggcgct

SEQ ID NO: 40 - DNA, 18 bp, Artificial
  Description of Artificial Sequence Synthetic primer
agcgccaccattcagtgc

SEQ ID NO: 41 - DNA, 19 bp, Artificial
  Description of Artificial Sequence Synthetic primer
actctgactcacagtacgc

SEQ ID NO: 42 - DNA, 19 bp, Artificial
  Description of Artificial Sequence Synthetic primer
gcgtactgtgagtcagagt

SEQ ID NO: 43 - DNA, 18 bp, Artificial
  Description of Artificial Sequence Synthetic primer
cagcgcctgacagcagtg

SEQ ID NO: 44 - DNA, 18 bp, Artificial
  Description of Artificial Sequence Synthetic primer
cactgctgtcaggcgctg

SEQ ID NO: 45 - DNA, 18 bp, Artificial
  Description of Artificial Sequence Synthetic primer
tggcagcagtgacgtctg

SEQ ID NO: 46 - DNA, 18 bp, Artificial
  Description of Artificial Sequence Synthetic primer
cagacgtcactgctgcca

SEQ ID NO: 47 - DNA, 26 bp, Artificial
  Description of Artificial Sequence Synthetic primer
gtaataagcgttgacaatttaaccgc

SEQ ID NO: 48 - DNA, 26 bp, Artificial
  Description of Artificial Sequence Synthetic primer
gcggttaaattgtcaacgcttattac

SEQ ID NO: 49 - DNA, 27 bp, Artificial
  Description of Artificial Sequence Synthetic primer
ttctttcacagatgtgaattggcgata

SEQ ID NO: 50 - DNA, 27 bp, Artificial
  Description of Artificial Sequence Synthetic primer
tatcgccaattcacatctgtgaaagaa

SEQ ID NO: 51 - DNA, 29 bp, Artificial
  Description of Artificial Sequence Synthetic primer
aattaaaaaactcttccagattttctcgg

SEQ ID NO: 52 - DNA, 34 bp, Artificial
  Description of Artificial Sequence Synthetic primer
aattaaaaacatctgaacttcagcctccagtaca

SEQ ID NO: 53 - DNA, 17 bp, Artificial
  Description of Artificial Sequence Synthetic primer
agccagcgcggatcatc

SEQ ID NO: 54 - DNA, 18 bp, Artificial
  Description of Artificial Sequence Synthetic primer
ctgtgaagcgccgaaatc

SEQ ID NO: 55 - DNA, 18 bp, Artificial
  Description of Artificial Sequence Synthetic primer
gtctgggactgggtggat

SEQ ID NO: 56 - DNA, 23 bp, Artificial
  Description of Artificial Sequence Synthetic primer
attacatgactcgaggtcgacgg